(19) United States
(12) Patent Application Publication
(10) Pub. No.: US 2010/0279952 A1
(43) Pub. Date: Nov. 4, 2010

(54) CROSS-LINKED HYDROGEL CONTAINING AN ACTIVE SUBSTANCE

(76) Inventors: Ninus Caram-Lelham, Uppsala (SE); Ulf Winter, Uppsala (SE); Bengt Agerup, Paris (FR)

Correspondence Address: BIRCH STEWART KOLASCH & BIRCH, PO BOX 747, FALLS CHURCH, VA, 22040-0747, US

(21) Appl. No.: 12/747,435

(22) PCT Filed: Dec. 22, 2008
(86) PCT No.: PCT/SE08/51550

§ 371 Date: Jun. 10, 2010

(30) Foreign Application Priority Data
Dec. 21, 2007 (SE) 0702904-4

(51) Int. Cl. A61K 38/14; A61K 47/36; A61K 47/38; C07K 14/00; C08B 1/00; C08B 31/00; C08B 37/02; C07H 1/00

(52) U.S. Cl. 514/20.9; 514/777; 514/778; 514/781; 530/395; 536/56; 536/102; 536/112; 536/123.1

(57) ABSTRACT

The invention relates to a biocompatible metastable intermediate material for controlling the mobility of at least one biologically active substance. An example of the invention is a hydrogel formed by cross-linked sodium hyaluronate treated with an oxidizing agent so as to open sugar rings and form aldehyde groups. The gel according to the invention is sterilized, e.g. by autoclaving.

### FIELD OF THE INVENTION

The present invention relates to a biocompatible metastable intermediate material, such as a gel, for controlling the mobility of biologically active substances, wherein the intermediate material is a cross-linked, activated, sterilized, preferably autoclaved polysaccharide. The invention also relates to a method for preparation of said intermediate material, and the use thereof as a medical device and for drug delivery. Furthermore the present invention relates to a pharmaceutical delivery system comprising at least one metastable intermediate material and at least one sterile biologically active substance, a method for preparation of the pharmaceutical delivery system and the use thereof for administration of biologically active substances. Also the invention relates to a kit comprising at least two components of which the first is a metastable intermediate material or a pharmaceutical delivery system and the second is a biologically active substance.

### BACKGROUND OF THE INVENTION

Many demands are made on pharmaceutical and medical products in order for them to be regarded as safe, one such demand being sterility. Heat, radiation or gas, or combinations thereof, are primarily used to confer sterility to a product by killing off unwanted components such as spores, bacteria and viruses or elements that could cause undesired growth of these components. Secondly sterilizing filters are used to separate active substance from unwanted components.

Biologically active substances such as proteins and peptides would denature or become inactivated from heat, radiation or gas treatment. They thus have to be sterilized using sterilizing filters, a means of sterilization not applicable to cross-linked polysaccharides, such as gels, due to their size. Hence a pharmaceutical product comprising a polysaccharide carrier such as a gel, and an active substance such as a protein or a peptide cannot be sterilized in form of the final product. All components comprised in the pharmaceutical product consequently have to be sterile before they are combined.

In general biologically active substances requiring sterile filtration are supplied as solids to be reconstituted by means of sterile solvents, usually sterile water for intravenous use. It is difficult to control the mobility and release of the active substance by administrating such an aqueous solution.

There are known means for controlling the release of biologically active substances, such as Lupron Depot (TAP Pharmaceutical Products Inc. Illinios, USA), a polylactide-co-glycolide microsphere formulation which releases gonadotropin releasing hormone analogue for treatment of prostate cancer and endometriosis, the implantable contraceptive Norplant (registered trademark of the Population Council) which comprises a silicone rubber tube filled with a steroid dispersion, wherein drug release is controlled by permeability of the steroid in the tube wall and remains fairly constant over several years, and drug delivery coating on stents, wherein the drug is incorporated in the coating and is released in a specific manner to achieve a certain pharmacological effect such as reduction of restenosis(http://www.ptca.org/des.html).

Water-binding gels of polysaccharides are widely used in the biomedical field. They are generally prepared by chemical cross-linking of polymers to infinite networks. One of the most widely used biocompatible polymers for medical use is hyaluronic acid, a polysaccharide. As it is present in identical composition in each living organism, it gives a minimum of side reactions and allows for advanced medical uses. Other biocompatible polysaccharides are e.g. dextran, alginate and heparin.

In the review article by N. Kashyap, N. Kumar and M. N. V. Ravi. Kumar in Critical Reviews in Therapeutic Drug Carrier Systems, 22(2):107-149 (2005), “Hydrogels for pharmaceutical and biomedical applications” hydrogels are described with their advantages and disadvantages. Advantages of hydrogels are e.g. their excellent tissue compatibility, easy manipulation and solute permeability. Some significant limitations of hydrogels represent their disadvantages, such as low mechanical strength, difficulty to sterilize and toxicity posed by the cross-linkers.

James R. Glass, et al. describe in Biomaterials, vol 17, pages 1101-1108, (1996), “Characterization of hyaluronic acid-Arg-Gly-Asp peptide cell attachment matrix” the use of BDDE (1,4-butanediyl diglycidyl ether) to crosslink hyaluronan and sodium periodate to form aldehyde groups.

U.S. 2004/0077592 discloses a biodegradable carrier for delivery of therapeutic agents, wherein the carrier comprises cross-linked polysaccharides. The carrier is prepared by reacting a first polysaccharide derivative having aldehyde groups with a second polysaccharide amine derivative, whereupon the carrier adapts a gel-like or sponge-like form as the two polysaccharides cross-link. A therapeutic agent can be entrapped within the gel/sponge either by mixing the agent with one of the deriviatives before gelatinization, or diffusion from a drug solution. The agent can also be covalently linked to the carrier prior to forming a gel or sponge by reacting aldehyde groups on the carrier with amine groups on the agent. Ways of making the end product sterile are not mentioned but it can be assumed that all components for preparing the carrier must be sterile before mixing and reaction since sterilization after these steps is not an option.

In U.S. 2007/0149441 (Aeschlimann et al) there is described a method for chemical modification of hyaluronic acid with various functional groups that allow for cross-linking of the hyaluronic acid under physiological conditions. In this application it is recognized that a process comprising activation of a hyaluronic acid to provide aldehyde groups, e.g. by using sodium periodate, and combining it with native HYA and some bio-active molecules, will reduce the biocompatibility of the final cross-linked gel due to a loss of native backbone structure which render it non-recognizable by the cells. Aeschlimann et al. in '441 solves this problem with a relatively complicated process comprising the introduction of various functional groups to bring about the desired cross-linking and binding of biomolecules.

In the article by Robert A. Peattie, et al., Biomaterials, vol 27, issue 9, pages 1868-1875, (2006), “Dual growth factor-induced angiogenesis in vivo using hyaluronan hydrogel implants” there is described the use of hyaluronan as protein/peptide carrier or matrix, wherein all steps of the reaction were carried out under aseptic conditions, and the films thus prepared were stored aseptically until needed.

There is still a need for an improved sterile delivery means that controls the mobility of biologically active substances after administration thereof. Likewise there is a need for an improved method for preparation of a pharmaceutical delivery system comprising biologically active substance.

### SUMMARY OF THE INVENTION

The present invention at least partly overcome the shortcomings of the prior art methods by providing a biocompatible sterile metastable intermediate material, which can be used for controlling the mobility of biologically active substances. The skilled man knew at the time of the application that it is possible to activate polysaccharides with aldehyde groups. Such groups when present may covalently bind amine groups of biologically active substances. However, it was thought that such gels could not be sterilized by autoclaving without losing the activity, i.e. by losing aldehyde groups and/or without degradation of the gel to the extent that the gel properties are lost.

In a first aspect of the invention there is provided a biocompatible metastable intermediate material, wherein the intermediate material is a cross-linked, activated, sterilized, preferably autoclaved polysaccharide comprising aldehyde groups for binding of biologically active substances to a desired degree.

In a second aspect the invention provides a method for preparation of the metastable intermediate material.

In another aspect of the invention there is provided the use of the metastable intermediate material as a medical device and for drug delivery.

In still another aspect the invention provides a biocompatible pharmaceutical delivery system for controlling mobility of at least one biologically active substance, such as for tissue regeneration, which system comprises at least one metastable intermediate material of the invention and at least one sterile biologically active substance.

In a further aspect of the invention there is provided a method for preparation of the pharmaceutical delivery system comprising at least one metastable intermediate gel of the invention and at least one sterile biologically active substance.

In yet another aspect of the invention there is provided the use of said system for administration of a biologically active substance.

Furthermore, in one aspect of the invention a kit is provided comprising at least two components of which the first is a metastable intermediate material of the invention or a pharmaceutical delivery system of the invention and the second is a biologically active substance, preferably sterile.

### DETAILED DESCRIPTION OF THE INVENTION

The present invention relates in a first aspect to a biocompatible metastable intermediate material for controlling the mobility of biologically active substances. The metastable intermediate material is a cross-linked, activated, sterilized, preferably autoclaved polysaccharide comprising aldehyde groups for binding of biologically active substances. The metastable intermediate material is insoluble in aqueous solution.

In the present application certain terms and expressions are given following certain meanings:

The term “activated” is used to describe a property of a cross-linked polysaccharide material in that it contains functional groups (aldehydes) intended for reaction with functional groups (amines) on other molecules, such as proteins and peptides. The term “active aldehyde groups” should be construed to mean aldehyde groups contained in the material intended for use and reactive in the covalent binding of amine groups on other molecules such as proteins and peptides. Hence active aldehyde groups in the sense of the present invention are not used for cross-linking.

By “metastable” we mean material that can maintain its properties upon storage and still remain reactive towards certain molecules.

By “gel” we mean a porous network of interconnected molecules, such as polysaccharide molecules, that spans the volume of a liquid medium and is insoluble in the liquid medium.

By “autoclaving” we mean steam sterilization.

By “biologically active substances” we mean compounds containing amine groups, such as proteins and peptides.

The polysaccharide of the metastable intermediate material is selected from the group consisting of dextran, alginate, chitosan, starch, cellulose, hyaluronic acid and other glucoseaminoglycans and their derivates, preferably hyaluronic acid.

The term hyaluronic acid can be interchangeably replaced with hyaluronan, wherein hyaluronic acid exists in several salt forms such as sodium hyaluronate.

The metastable intermediate material according to the first aspect of the invention is characterized by being biocompatible, cross-linked, activated and sterilized, preferably autoclaved. This enables the material, such as a gel, being used as carrier or matrix for a biologically active substance that is to be administered e.g. subcutaneously or intramuscularly to a subject, or implanted in a subject. Since it is well-known that hyaluronan degrades when it is autoclaved and degrades also when oxidized using sodium periodate, it is surprising that a low-degree-cross-linked material of hyaluronan can be both oxidized and autoclaved and still remain activated, maintaining its biocompatibility and form. A highly cross-linked hyaluronan gel may withstand oxidization and autoclaving , but most probably will lead to side effects due to the high degree of modification of the molecule and thereby less recognition by the living cells.

Preferably the metastable intermediate material is a gel. It may be particulate or constitute a film. In particulate form it can easily be injected by means of a syringe. The metastable intermediate material may also be in form of another physical entity made from polysaccharides, such as a cardiac valve, or be coated on a physical entity in such a way that the entity becomes biocompatible.

Since a metastable intermediate material produced according to the invention is sterilized, preferably autoclaved, and may be stored for a certain period of time without significant loss of activity, i.e. loss of active aldehyde groups , or any other characteristics of the material, it is possible to store and transport it to the final manufacturer or user.

From a manufacturing point of view, the present invention significantly simplifies the manufacturing process, increases the sterility assurance and decreases the cost for preparation of a pharmaceutical delivery system compared with an aseptically prepared system.

The metastable intermediate material of the invention has active aldehyde groups being able to react with compounds having amine groups, such as peptides and proteins. A further characteristic of the metastable intermediate material is its ability of remaining at the administration site for a prolonged time allowing a biologically active substance to act locally, e.g. in the eyes and knees, at the site of a surgery or in the cancer tissue, thereby exhibiting extended release or slow release,

According to a second aspect the present invention relates to a method for preparation of the metastable intermediate material, such as a gel, comprising the steps of:
a) cross-linking a polysaccharide to form a material, such as a gel, or providing a cross-linked polysaccharide, e.g. in gel form;
b) treating the cross-linked polysaccharide material with an oxidizing agent so as to open sugar rings and form active aldehyde groups;
c) removing and/or neutralizing unreacted cross-linking agent, if any, and the oxidizing agent;
d) subjecting the gel to sterilization.

The sterilization of step d) is preferably performed by autoclaving.

The man skilled in the art knows how to cross-link a polysaccharide to form a material, such as a gel. Preferably the cross-linking is performed using a NaOH solution containing BDDE (1,4-butanediyl diglycidyl ether), a well known cross-linker. Cross-linked gels are also commercially available from companies such as Q-Med AB (Sweden). It is of no relevance for the method of the invention whether the gel is initially autoclaved or not. The importance lies in adjusting the initial degree of cross-linking to the subsequent treatment of the material, such as a gel, however with caution so as to retain its biocompatibility

The formation of aldehyde groups is achieved by using an oxidizing agent, preferably sodium periodate by reaction of polysaccharide and sodium periodate. The amount of oxidizing agent used is dependent on the amount of gel, the desired number of active aldehyde groups (degree of activation) and time of reaction. Normally the reaction is carried out at room temperature, it is however possible to perform the reaction at other temperatures. It is stressed that the purpose of forming aldehyde groups is to enable to covalently bind biologically active molecules to the metastable intermediate material, not to use them for any cross-linking.

In a rinsing step unreacted cross-linking agent, if any, and oxidizing agent are removed by washing using aqueous solution(s) containing salt, buffer, ethanol or combinations of these. Preferably deionized water or a solution of 0,9% NaCl are used in the rinsing step.

Autoclaving is suitably performed by steam sterilization using standard procedures common in medical device and pharmaceutical industry. Preferably autoclaving is performed at 121° C. for 20 minutes.

According to the present invention the metastable intermediate material can be produced in a reproducible manner and can be characterized and well-documented prior further use. The present invention also allows producing materials having different cross-linking and activation degrees that can be used for different purposes. Combinations of gels with different cross-linking and activation degrees may be combined to achieve desired duration and/or activity.

In another aspect the present invention relates to the use of a metastable intermediate material, such as a gel, as a medical device and for drug delivery. Typical examples of such medical devices are stents, catheters, soft tissue implants and cardiac valves. For drug delivery the material may be used as a carrier or matrix for delivery of vaccines and drugs, such as anticancer drugs, toxins, growth factors and antibiotics.

According to yet another aspect the present invention relates to a biocompatible, pharmaceutical delivery system for controlling mobility of at least one biologically active substance, comprising at least one metastable intermediate material, such as a gel, and at least one sterile biologically active substance, wherein the biologically active substance is covalently bound to the metastable intermediate gel. The system may comprise metastable intermediate materials, such as gels, having different cross-linking degrees and/or different activation degrees, i.e. different amounts of active aldehyde groups for binding of amine groups on biologically active substances, such as proteins and peptides. Examples of biologically active substances are growth factors, cancer drugs, anti-inflammatory drugs, toxins and antigens. It is also conceivable that the gel in addition to any covalently bound substances contains at least one non-bound biologically active substance to be used for boost dosage after administration.

Since many proteins and peptides are very expensive, the use of the metastable intermediate material, such as a gel, of the invention with its improved characteristics minimizes the risk for failure and therefore production costs are reduced.

The present invention relates in a further aspect to a method for preparation of said pharmaceutical delivery system comprising the step of bringing at least one sterile biologically active substance in contact with at least one metastable intermediate material, optionally in the presence of a reducing agent for further reduction, thereby binding a biologically active substance covalently to the active aldehyde groups of the metastable intermediate gel through Schiff base formation.

The use of a reducing agent will convert the bonds between the biologically active substance and the metastable intermediate material into bonds that are highly stable and not readily hydrolysable in aqueous environment. The conversion of said bonds by the use of reducing agent is due to a shift of chemical equilibrium towards bonds that cannot be hydrolysed and consequently the biologically active substance will be firmer bound to the metastable intermediate material. Reducing agents include e.g. cyanoborohydride, sodium borohydride and ascorbic acid, wherein ascorbic acid is preferred in the present invention. Using ascorbic acid will give a ready-to-use product without any further purification.

Preferably the reducing agent is added upon mixing the biologically active substance and the metastable intermediate gel or after the mixing has occurred.

The more amine groups contained in a biologically active substance, the higher the number of binding sites to the gel and the lesser the need of using a reducing agent.

In a further aspect the present invention relates to the use of said system for administration of biologically active substance. One important feature of the present invention is the possibility to provide localized deposition of a large variety of substances, that if administered by e.g. intravenous methods would cause undesirable systemic effects. This is particularly the case where the substances are toxic in some respect, and the toxicity is to be used for local treatment at a specific trauma site in the body. A typical example would be toxins for the treatment of e.g. cancer tumours.

The use of biocompatible metastable intermediate materials with different cross-linking and activation degrees, in combination with thereto bound biologically active substances, wherein the resulting aldehyde-amine bonds show different hydrolysability, will provide a system with a desired release profile of biologically active substances. A polysaccharide material with low cross-linking degree would degrade faster once administered into the body of a subject than a polysaccharide material with high cross-linking degree. A biologically active substance would consequently be released faster from the metastable intermediate material if bound to a material having low degree of cross-linking, e.g. as a boost dose, due to the body's ability to faster degradation thereof, than if bound to a material having high degree of cross-linking. By degradation it is meant the breakdown of the metastable intermediate material by processes in the body. The activation degree of a metastable intermediate material also render it possible to control the desired release profile of a biologically active substance since the more aldehyde groups present the higher the number of binding sites for amine groups in the biologically actice substance. The hydrolysability of the bonds between aldehydes of the metastable intermediate material and amines of the biologically active substance also gives a mechanism for controlling the release of active substance.

The system may for example be used for tissue regeneration, e.g. comprising the growth factor BMP (bone morphogenic protein) bound to the metastable intermediate material, wherein BMP will be released after administration to a subject as the metastable intermediate material, such as a gel, degrades in the body. The system may for example also be used in a vaccine comprising antigens bound to the metastable intermediate material.

The ability of the gel to bind substances with amine functionality, e.g. peptides and proteins, or hormones, will open up an entire field of applications with potentially superior behaviour compared with currently used methods.

It has also very large economic implications, in that the dose rate can be substantially reduced with a depot type of administration. In clinical studies of prior art methods it has been possible to show a reduction of the rate from two doses per week to one dose per month, i.e. almost 90% reduction. For e.g. growth hormone or hormones associated with anaemia, which are very expensive medicaments, this will mean significant savings.

A further great advantage of binding proteins and other complex substances to a material according to the present invention, such as a gel, is that such substances can be used virtually in their native state, i.e. no modifications of the compounds themselves will be necessary. The only “modification” will be that some functionalities will be used for the binding to the gel, but such bonds will not affect the substance, once it has been released from the gel.

According to still another aspect of the invention a kit is provided comprising at least two component of which the first is a metastable intermediate material of the invention or a pharmaceutical delivery system of the invention and the second is a biologically active substance. The metastable intermediate material may be a gel, a film or a physical entity made up of said material or coated with said material. The pharmaceutical delivery system comprises at least one metastable intermediate material and at least one biologically active substance bound to the gel. Furthermore a non-bound biologically active substance may be present in the metastable intermediate material, wherein it may be introduced into the material e.g. by diffusion.

It is conceivable to provide a kit, wherein e.g. the metastable intermediate material or pharmaceutical delivery system may be provided in a vial and the biologically active substance in a syringe for mixing before use. Another possible kit may include two syringes comprising the metastable intermediate material or pharmaceutical delivery system and the biologically active substance respectively, wherein the mixing is performed by connecting the two syringes and mix their content by alternate emptying the one syringe into the other.

The kit of the invention can maintain its properties upon storage for at least six months, preferably longer.

### EXAMPLES

### Example 1

### (Preparation of an Activated Gel)

Restylane Sub Q (manufactured by and obtainable from Q-Med, Uppsala, Sweden) was washed with 0.9% NaCl solution using a Buchner funnel. About 9.7 g of the washed gel was mixed with 9,7 g of a solution of the oxidizing agent sodium periodate (2.3 mM in 0.9% NaCl) for 2 minutes. The gel was washed repeatedly using 0.9% NaCl and then autoclaved at 121° for 20 minutes. Using BCA (bicinchoninic acid; protein assay reagent obtainable from Pierce) it was shown that the gel contained aldehyde groups by changing colour. The washed Sub Q gel that was neither treated with sodium periodate nor autoclaved did not show sign of measurable amount of aldehyde groups.

### Example 2

### (Preparation of an Activated Gel and Test for Activity)

As an oxidizing agent, sodium periodate was added to Restylane Sub Q (manufactured by and obtainable from Q-Med, Uppsala, Sweden) which is a gel comprised of cross-linked hyaluronic acid. After about 40 minutes the mixture was washed several times using deionized water. in order to test for aldehyde activity, an aqueous solution of fuchsin was added to the washed gel. After about 25 minutes the gel was washed with 0.9% NaCl solution repeatedly. The gel was deep red indicating that fuchsin was attached to the gel due to the presence of aldehyde groups. Gel not treated with sodium periodate was only slightly colored.

### Example 3

### (Preparation of a Cross-Linked Gel and Effect of Varying Amounts of Oxidizing Agent on Activation)

Sodium hyaluronate was cross-linked using BDDE to form a gel. Gel pieces were treated with different amount of sodium periodate (ranging from 0 to 40 mg) for 40 minutes at room temperature and then washed extensively using 0.9% NaCl. Using BCA (protein assay reagent by Pierce) it was shown by change in colour that the amount of aldehyde groups found in the gel pieces increased with increasing amount of sodium periodate.

### Example 4

### (Autoclaving of Gels Activated to Different Degrees)

A set of gel pieces prepared according to example 3 was autoclaved at 121° C. for 20 minutes. The samples treated with 30 and 40 mg sodium periodate behaved as a viscous solution rather than gel particle. However, the rest of the samples activated with sodium periodate (i.e. at concentrations? 10 and 20 mg) were still in the gel form and according to the BCA remained activated, containing aldehyde groups.

### Example 5

### (Binding of Protein to an Activated Gel Compared to a Non-Activated Gel)

A FITC-albumin solution was added to one activated and one non-activated hyaluronan gel in phosphate buffer. The activated gel was clearly yellow indicating FITC-albumin attachment to the gel. The non-activated gel was clear with almost no yellow color.

### Exampel 6

(Binding of Protein to Autoclaved Gels with Different Activation Degrees)

A FITC-albumin solution was added to each of activated and autoclaved gels produced in example 4. The gel treated with 20 mg sodium periodate was most yellow and gel treated with 0.4 mg sodium periodate was least yellow.

1-20. (canceled)

21. A biocompatible metastable intermediate material for controlling the mobility of at least one biologically active substance, characterized in that
said metastable intermediate material is a cross-linked, sterilized polysaccharide comprising aldehyde groups capable of binding biologically active substances to a desired degree,
said polysaccharide is selected from the group consisting of dextran, alginate, chitosan, starch, cellulose, hyaluronic acid and other glucoseaminoglycans and their derivates, and in that
said material has been sterilized by autoclaving.

22. Metastable intermediate material according to claim 21, characterized in that said polysaccharide is hyaluronic acid.

23. Metastable intermediate material according to claim 21, characterized in that the material is a gel.

24. Metastable intermediate material according to claim 21, characterized in that the material is a physical entity at least partially made up of said material.

25. Metastable intermediate material according to claim 21, characterized in that the material is a physical entity made up entirely of said material.

26. A method for preparation of the metastable intermediate material according to claim 21, comprising the steps of:
cross-linking a polysaccharide to form a material, or providing a cross-linked polysaccharide material;
treating the cross-linked polysaccharide material with an oxidizing agent so as to open sugar rings and form aldehyde groups;
removing and/or neutralizing unreacted cross-linking agent and oxidizing agent; and subjecting the gel to autoclavation.

27. The use of a metastable intermediate material according to claim 21, as a medical device and for drug delivery.

28. A biocompatible pharmaceutical delivery system for controlling mobility of at least one biological substance comprising at least one metastable intermediate gel according to claim 21, and at least one sterile biologically active substance which is covalently bound to said metastable intermediate material via aldehyde groups in the material.

29. System according to claim 28, characterized in that said biologically active substance is a protein or a peptide.

30. System according to claim 28 or 29, characterized in that said biologically active substance is sterilized.

31. A method for preparation of the system according to claim 28, comprising the step of bringing said biologically active substance in contact with said metastable intermediate material, wherein said biologically active substance binds covalently to said metastable intermediate material via aldehyde groups in the material.

32. Method according to claim 31, wherein the binding of said biologically active substance to said metastable intermediate gel via aldehyde groups in the material takes place in the presence of a reduction agent.

33. Method according to claim 32, wherein said reduction agent is cyanoborohydride or sodium borohydride.

34. Method according to claim 32, wherein said reduction agent is ascorbic acid.

35. The use of the system according to claim 28, for administration of biologically active substances.

36. Kit comprising at least two components, comprising;
a metastable intermediate material according to claim 21, or a system according to claim 28; and
a biologically active substance.

37. Kit according to claim 36, characterized in that the biologically active substance is sterilized.
